(19) United States
(12) Patent Application Publication
(10) Pub. No.: US 2008/0319209 A1
(43) Pub. Date: Dec. 25, 2008

(54) Process for Preparing (Disubstitutedpropenyl) Phenylalkyl Substituted Dihydrobenzofurans

(76) Inventors: Guozhi Wang, Freshmeadow, NJ (US); Charles E. Hatch III, Pennington, NJ (US); Jaidev S. Goudar, West Windsor, NJ (US); Russell Patera, Hamilton, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/816,486

(22) PCT Filed: Feb. 16, 2006
(86) PCT No.: PCT/US06/05345

§ 371 Date: Jul. 30, 2008

Related U.S. Application Data
(60) Provisional application No. 60/653,736, filed on Feb. 17, 2005.

(51) Int. Cl. C07F 9/06; C07D 307/00; C07F 9/02

(52) U.S. Cl. 549/220; 549/469; 568/14

(57) ABSTRACT

An improved process is described for preparing (disubstitutedpropenyl) phenylalkyl substituted dihydrobenzofurans. This improved process is focused on steps to produce key intermediates, namely disubstitutedphenolylalkyl substituted dihydrobenzofurans of formula I:

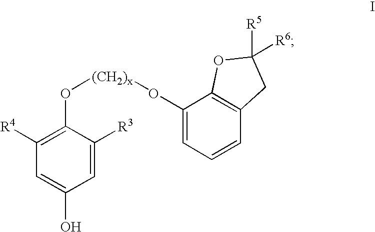

where R³, R⁴, R⁵, R⁶ and x are defined herein.

This application claims the benefit of U.S. Provisional Application No. 60/653,736, filed Feb. 17, 2005.

### FIELD OF THE INVENTION

This invention is in the field of chemical processes; more specifically, an improved process for preparing (disubstitutedpropenyl) phenylalkyl substituted dihydrobenzofurans.

### BACKGROUNDS

(Disubstitutedpropenyl) phenylalkyl substituted dihydrobenzofurans, such as:

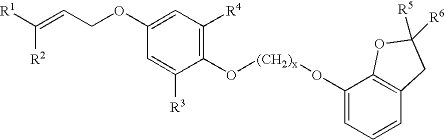

wherein R¹, R², R³, R⁴, R⁵ and R⁶ are independently selected from halogen or alkyl and x is 2, 3, 4, 5 or 6; are useful insecticides and have been described in U.S. Pat. No. 6,987,194, the disclosure of which is incorporated herein by reference. Disadvantages of processes to produce these compounds include less than optimal yields, less than optimal cycle times and high catalyst loadings. Compounds represented by formula I:

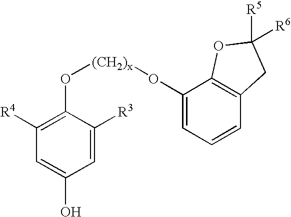

wherein R³, R⁴, R⁵, R⁶ and x are as defined above; are key intermediates in the process for preparing (disubstitutedpropenyl) phenylalkyl substituted dihydrobenzofurans.

### SUMMARY OF THE INVENTION

The present invention improves the process for preparing compounds of formula I. As a result of the present invention, overall yield, cycle times and catalyst loading are improved for the production of (disubstitutedpropenyl) phenylalkyl substituted dihydrobenzofurans. Specifically, it has now been found that a compound of formula I:

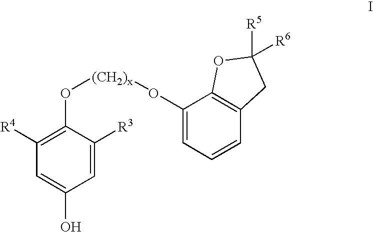

wherein
R³ and R⁴ are selected from halogen;
R⁵ and R⁶ are independently selected from halogen or alkyl; and
x is2, 3, 4, 5 or 6;
   can be prepared in excellent yield and purity by a process comprising:

a) reacting a compound of formula (A):

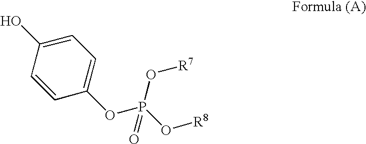

wherein R⁷ and R⁸ are independently selected from hydrogen, alkyl, aryl or R⁷ and R⁸ taken together with an alkyl or aryl, forming a cyclic ester;

with a halogenating agent in the presence of a base to form a compound of formula II:

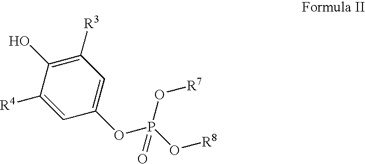

wherein
R³ and R⁴ are as defined above; and
R⁷ and R⁸ are as defined above;

b) reacting a compound of formula (B):

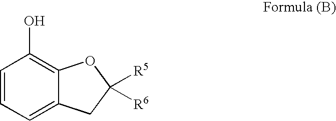

wherein R⁵ and R⁶ are as defined above;

with a compound of formula (C):

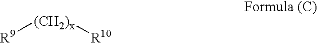

wherein
R⁹ and R¹⁰ are independently selected from halogen, hydroxyl or —OSO₂R¹¹
   wherein R¹¹ is alkyl or aryl; and
x is 2, 3, 4, 5 or 6;

in the presence of a base to form a compound of formula III:

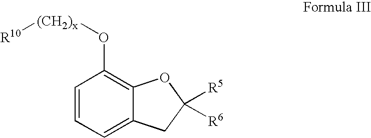

wherein
R⁵, R⁶ R¹⁰ and x are as defined above;

c) reacting a compound of formula II with a compound of formula III in the presence of a base to form a compound of formula IV:

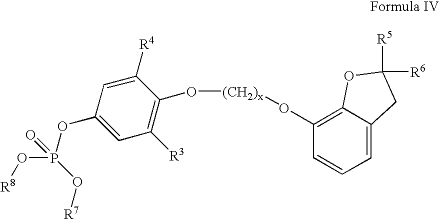

wherein
R³, R⁴, R⁵, R⁶, R⁷, R⁸ and x are as defined above; and

d) reacting a compound of formula IV to form a compound of formula I.

### Definitions

The modifier “about” is used herein to indicate that certain preferred operating ranges, such as ranges for molar ratios for reactants, material amounts, and temperature, are not fixedly determined. The meaning will often be apparent to one of ordinary skill. For example, a recitation of a temperature range of about 120° C. to about 135° C. in reference to, for example, an organic chemical reaction would be interpreted to include other like temperatures that can be expected to favor a useful reaction rate for the reaction, such as 105° C. or 150° C. Where guidance from the experience of those of ordinary skill is lacking, guidance from the context is lacking, and where a more specific rule is not recited below, the “about” range shall be not more than 10% of the absolute value of an end point or 10% of the range recited, whichever is less.

As used in this specification and unless otherwise indicated the substituent terms “alkyl”, “alkoxy”, and “haloalkyl”, used alone or as part of a larger moiety, includes straight or branched chains of at least one or two carbon atoms, as appropriate to the substituent, and preferably up to 12 carbon atoms, more preferably up to ten carbon atoms, most preferably up to seven carbon atoms. “Halogen”, “halide” or “halo” refers to fluorine, bromine, iodine, or chlorine. The term “ambient temperature” refers to a temperature in the range of about 20° C. to about 30° C. Certain solvents, catalysts, and the like are known by their acronyms. These include the acronyms “DMAC” meaning N,N-dimethylacetamide, “DMF” meaning N,N-dimethylformamide, “THF” meaning tetrahydrofuran. The term “glymes” refers to a class of solvents comprised of monoglyme, diglyme, triglyme, tetraglyme, and polyglyme. The term “GC” refers to gas chromatography or gas chromatographic methods of analyses.

### DETAILED DESCRIPTION OF THE INVENTION

The present invention relates to a process for preparing a compound of formula I:

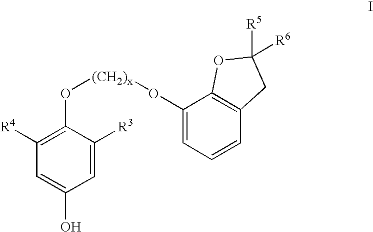

wherein
R³ and R⁴ are selected from halogen;
R⁵ and R⁶ are independently selected from halogen or alkyl; and
x is 2, 3, 4, 5 or 6;

said process comprising:

a) reacting a compound of formula (A):

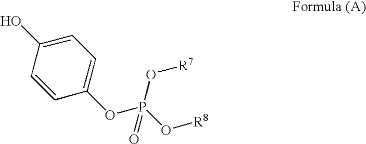

wherein R⁷ and R⁸ are independently selected from hydrogen, alkyl, aryl or R⁷ and R⁸ taken together with an alkyl or aryl, forming a cyclic ester;

with a halogenating agent in the presence of a base to form a compound of formula II:

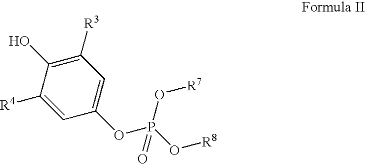

wherein
R³ and R⁴ are as defined above; and
R⁷ and R⁸ are as defined above;

b) reacting a compound of formula (B):

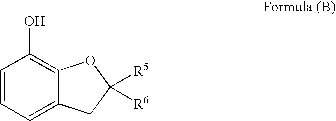

wherein R⁵ and R⁶ are as defined above;

with a compound of formula (C):

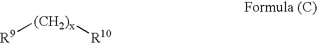

wherein
R⁹ and R¹⁰ are independently selected from halogen, hydroxyl or —OSO₂R¹¹
   wherein R¹¹ is alkyl or aryl; and
x is 2, 3, 4, 5 or 6;

in the presence of a base to form a compound of formula III:

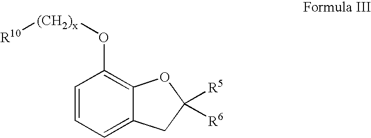

wherein
R⁵, R⁶ R¹⁰ and x are as defined above;

c) reacting a compound of formula II with a compound of formula III in the presence of a base to form a compound of formula IV:

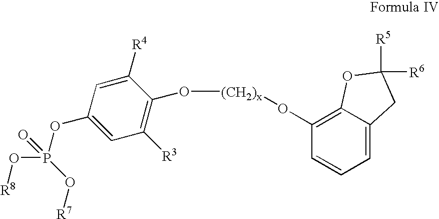

wherein
R³, R⁴, R⁵, R⁶, R⁷, R⁸ and x are as defined above; and

d) reacting a compound of formula IV to form a compound of formula I.

The reaction of step b) can be conducted in the presence of a catalyst; at elevated temperature. The catalyst can be polyethylene glycol, dimethylaminopyridine, triethylamine, p-toluenesulfonic acid, phosphorous pentoxide, pyridine, phase transfer catalysts such as quaternary ammonium salts or quaternary phosphonium salts or mixtures thereof. The catalyst can be present in a concentration of from about 0.1% by weight to about 15% by weight. The elevated temperature can be in the range of 30° C. to 120° C.

The reaction of step c) can be conducted in the presence of a solvent; in the presence of a catalyst; at elevated temperature. The solvent can be tetrahydrofuran, toluene, xylene, acetone, acetonitrile, 1,2-dichloroethane, triethylamine, p-dioxane, N,N-dimethylacetamide, N,N-dimethylformamide, glymes, methyl isobutyl ketone, dimethylsulfoxide or mixtures thereof. The catalyst can be polyethylene glycol, dimethylaminopyridine, triethylamine, p-toluenesulfonic acid, phosphorous pentoxide, pyridine, phase transfer catalysts such as quaternary ammonium salts or quaternary phosphonium salts or mixtures thereof. The catalyst can be present in a concentration of from about 0.1% by weight to about 20% by weight. The elevated temperature can be in the range of 30° C. to 110° C.

The reaction of step d) can be conducted with a base; as a hydrolysis in the presence of an acid; in the presence of a solvent; in the presence of a catalyst. The solvent can be tetrahydrofuran, toluene, xylene, 1,2-dichloroethane, triethylamine, p-dioxane, N,N-dimethylacetamide, N,N-dimethylformamide, glymes, methyl isobutyl ketone, dimethylsulfoxide or mixtures thereof. The catalyst can be polyethylene glycol, dimethylaminopyridine, triethylamine, p-toluenesulfonic acid, phosphorous pentoxide, pyridine, phase transfer catalysts such as quaternary ammonium salts or quaternary phosphonium salts or mixtures thereof. The catalyst can be present in a concentration of from about 0.1% by weight to about 20% by weight.

Another embodiment of the present invention is a process for preparing a compound of formula I:

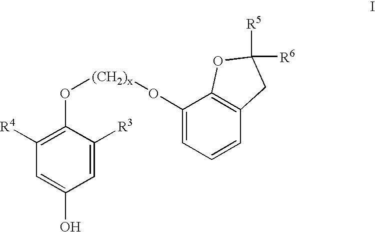

wherein
R³ and R⁴ are selected from halogen;
R⁵ and R⁶ are independently selected from halogen or alkyl; and
x is 2, 3, 4, 5 or 6;

said process comprising:

a) reacting a compound of formula II:

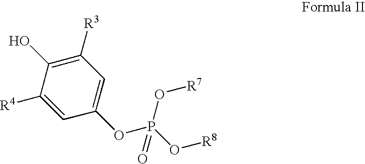

wherein
R³ and R⁴ are as defined above; and
R⁷ and R⁸ are independently selected from hydrogen, alkyl, aryl or R⁷ and R⁸ taken together with alkyl or aryl, forming a cyclic ester;

with a compound of formula (C):

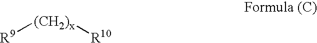

wherein
R⁹ and R¹⁰ are independently selected from halogen, hydroxyl or —OSO₂R¹¹
   wherein R¹¹ is alkyl or aryl; and
x is 2, 3, 4, 5 or 6;

in the presence of a base to form a compound of formula V:

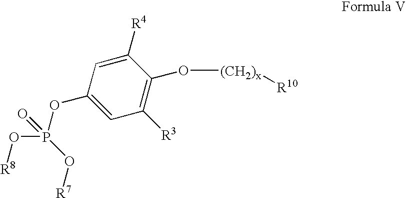

wherein
R³, R⁴, R⁷, R⁸, R¹⁰ and x are as defined above;

b) reacting a compound of formula V with a compound of formula (B):

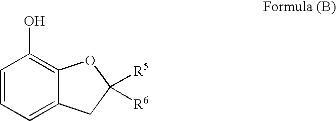

wherein R⁵ and R⁶ are as defined above;

in the presence of a base to form a compound of formula IV:

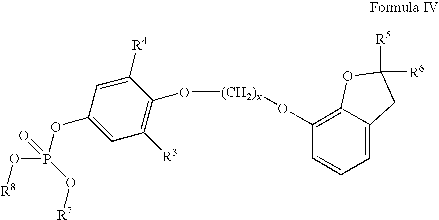

wherein
R³, R⁴, R⁵, R⁶, R⁷, R⁸ and x are as defined above; and

c) reacting a compound of formula IV to form a compound of formula I.

The reaction of step a) can be conducted in the presence of a catalyst; at elevated temperature. The catalyst can be polyethylene glycol, dimethylaminopyridine, triethylamine, p-toluenesulfonic acid, phosphorous pentoxide, pyridine, phase transfer catalysts such as quaternary ammonium salts or quaternary phosphonium salts or mixtures thereof. The catalyst can be present in a concentration of from about 0.1% by weight to about 15% by weight. The elevated temperature can be in the range of 30° C. to 100° C.

The reaction of step b) can be conducted in the presence of a solvent; in the presence of a catalyst; at elevated temperature. The solvent can be tetrahydrofuran, toluene, xylene, 1,2-dichloroethane, triethylamine, p-dioxane, N,N-dimethylacetamide, N,N-dimethylformamide, glymes, methyl isobutyl ketone, dimethylsulfoxide or mixtures thereof. The catalyst can be polyethylene glycol, dimethylaminopyridine, triethylamine, p-toluenesulfonic acid, phosphorous pentoxide, pyridine, phase transfer catalysts such as quaternary ammonium salts or quaternary phosphonium salts or mixtures thereof. The catalyst can be present in a concentration of from about 0.1% by weight to about 20% by weight. The elevated temperature can be in the range of 30° C. to 110° C.

The reaction of step c) can be conducted with a base; as a hydrolysis in the presence of an acid; in the presence of a solvent; in the presence of a catalyst. The solvent can be tetrahydrofuran, toluene, xylene, 1,2-dichloroethane, triethylamine, p-dioxane, N,N-dimethylacetamide, N,N-dimethylformamide, glymes, methyl isobutyl ketone, dimethylsulfoxide or mixtures thereof. The catalyst can be polyethylene glycol, dimethylaminopyridine, triethylamine, p-toluenesulfonic acid, phosphorous pentoxide, pyridine, phase transfer catalysts such as quaternary ammonium salts or quaternary phosphonium salts or mixtures thereof. The catalyst can be present in a concentration of from about 0.1% by weight to about 20% by weight.

Another embodiment of the present invention is a compound of formula II:

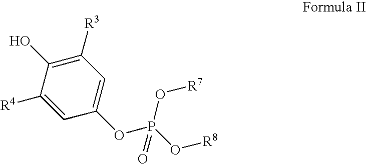

wherein
R³ and R⁴ are selected from halogen; and
R⁷ and R8 are independently selected from hydrogen, alkyl, aryl or R⁷ and R⁸ taken together with an alkyl or aryl, forming a cyclic ester.

Another embodiment of the present invention is a compound of formula III:

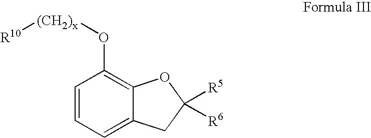

wherein
R⁵ and R⁶ are independently selected from halogen or alkyl;
R¹⁰ is selected from halogen, hydroxyl or —OSO₂R¹¹
   wherein R¹¹ is alkyl or aryl; and
x is 2, 3, 4, 5 or 6.

Another embodiment of the present invention is a compound of formula IV:

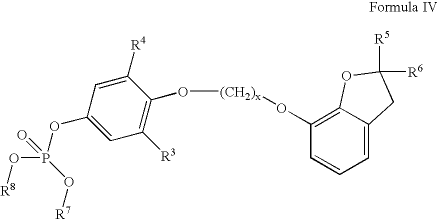

wherein
R³ and R⁴ are selected from halogen;
R⁵ and R⁶ are independently selected from halogen or alkyl;
R⁷ and R⁸ are independently selected from hydrogen, alkyl, aryl or R⁷ and R⁸ taken together with an alkyl or aryl, forming a cyclic ester; and
x is 2, 3, 4, 5 or 6.

Yet another embodiment of the present invention is a compound of formula V:

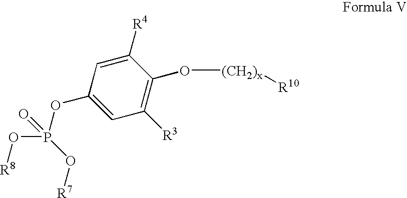

wherein
R³ and R⁴ are selected from halogen;
R⁷ and R⁸ are independently selected from hydrogen, alkyl, aryl or R⁷ and R⁸ taken together with an alkyl or aryl, forming a cyclic ester;
R¹⁰ is selected from halogen, hydroxyl or —OSO₂R¹¹
   wherein R¹¹ is alkyl or aryl; and
x is 2, 3, 4, 5 or 6.

The following examples illustrate processes for preparing compounds of formulae I, II, III, IV and V.

### Example 1

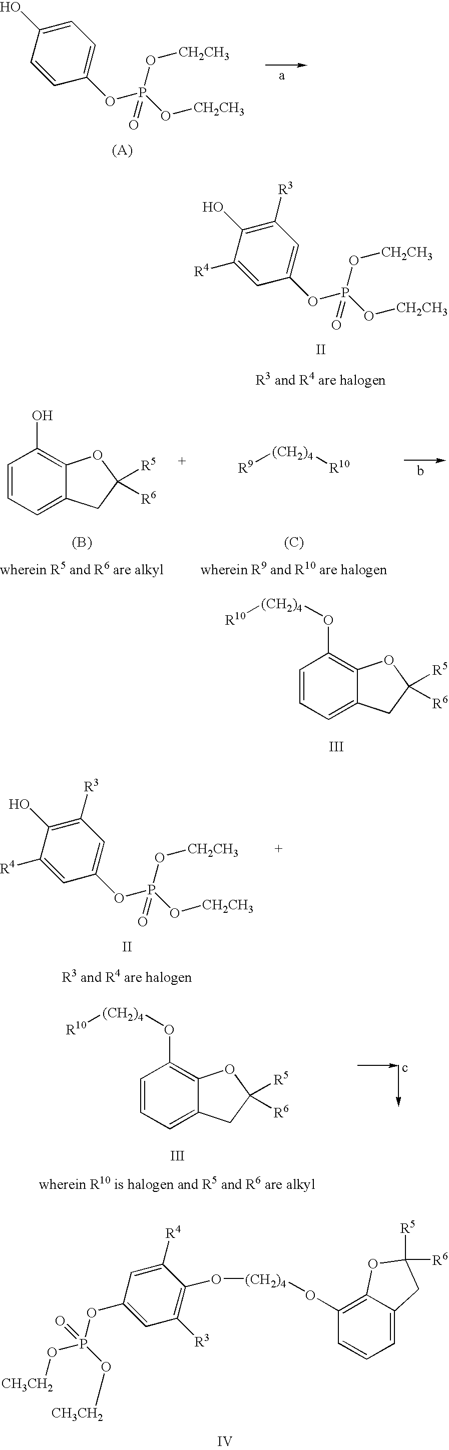

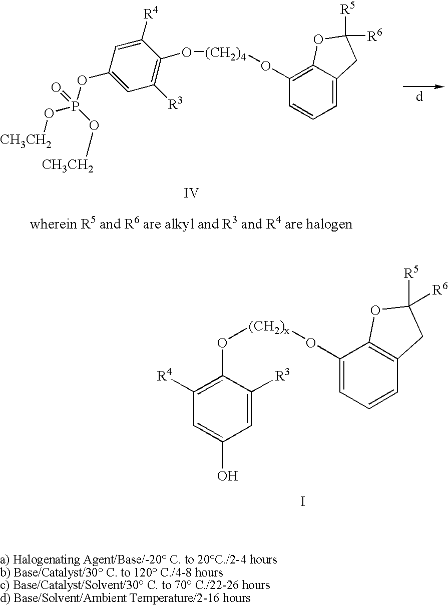

In the first step (a) of Example 1, dialkyl 4-hydroxybenzenephosphate (A), for example diethyl 4-hydroxybenzenephosphate, was reacted with a halogenating agent, for example sulfurylchloride, in the presence of a base at reduced temperature to form dialkyl 3,5-dihalo-4-hydroxybenzenephosphate, a compound of formula II, for example diethyl 3,5-dihalo-4-hydroxybenzenephosphate.

In step (b) of Example 1,2,2-dialkyl-2,3-dihydrobenzo[b]furan-7-ol (B) was reacted with a 1,4-dihaloalkane (C), for example 1,4-dihalobutane, in the presence of a base and a catalyst at elevated temperature to form a 1-(2,2-dialkyl(2,3-dihydrobenzo[2,3-b]furan-7-yloxy))-4-haloalkane, a compound of formula III, for example 1-(2,2-dialkyl(2,3-dihydrobenzo[2,3-b]furan-7-yloxy))-4-halobutane.

In step (c) of Example 1, dialkyl 3,5-dihalo-4-hydroxybenzenephosphate, a compound of formula II, for example diethyl 3,5-dihalo-4-hydroxybenzenephosphate, was reacted with a 1-(2,2-dialkyl(2,3-dihydrobenzo[2,3-b]furan-7-yloxy))-4-haloalkane, a compound of formula III, for example 1-(2,2- dialkyl(2,3-dihydrobenzo[2,3-b]furan-7-yloxy))-4-halobutane, in the presence of a base, a solvent and a catalyst at elevated temperature to form a dialkyl 4-[4-(2,2-dialkyl(2,3-dihydrobenzo[2,3-b]furan-7-yloxy))alkoxy]-3,5-dihalobenzenephosphate, a compound of formula IV, for example diethyl 4-[4-(2,2-dialkyl(2,3-dihydrobenzo[2,3-b]furan-7-yloxy))butoxy]-3,5-dihalobenzenephosphate.

In step (d) of Example 1, a dialkyl 4-[4-(2,2-dialkyl(2,3-dihydrobenzo[2,3-b]furan-7-yloxy))alkoxy]-3,5-dihalobenzenephosphate, a compound of formula IV, for example diethyl 4-[4-(2,2-dialkyl(2,3-dihydrobenzo[2,3-b]furan-7-yloxy))butoxy]-3,5-dihalobenzenephosphate, was reacted with a base at ambient temperature to form a 4-[4-(2,2-dialkyl(2,3-dihydrobenzo[2,3-b]furan-7-yloxy))alkoxy]-3,5-dihalophenol (I), for example 4-[4-(2,2-dialkyl(2,3-dihydrobenzo[2,3-b]furan-7-yloxy))butoxy]-3,5-dihalophenol.

### Example 2

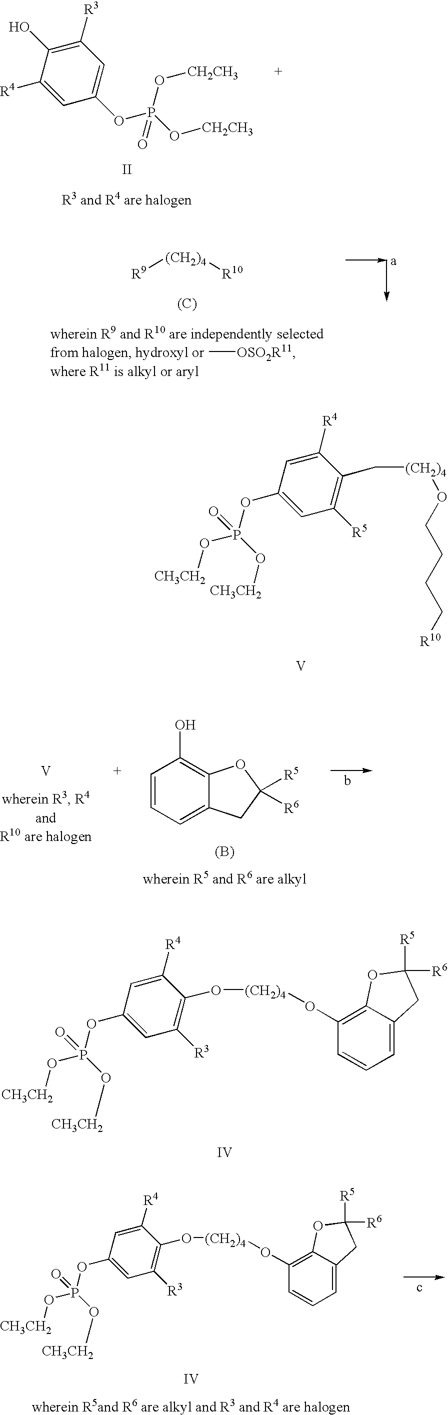

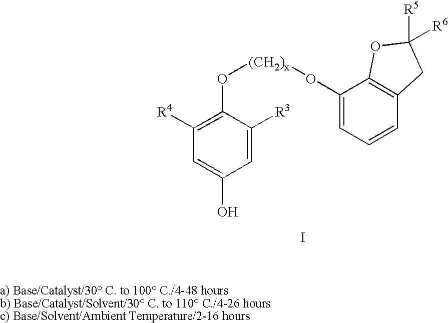

In the first step (a) of Example 2, dialkyl 3,5-dihalo-4-hydroxybenzenephosphate, a compound of formula II, for example diethyl 3,5-dihalo-4-hydroxybenzenephosphate, can be reacted with a 1,4-disubstitutedalkane (C), for example 1,4-disubstitutedbutane, in the presence of a base and a catalyst at elevated temperature to form dialkyl 4-(4-substitutedalkoxy)-3,5-dihalobenzenephosphate, a compound of formula V, for example diethyl 4-(4-substitutedbutoxy)-3,5-dihalobenzenephosphate.

In step (b) of Example 2, a dialkyl 4-(4-substitutedalkoxy)-3,5-dihalobenzenephosphate, a compound of formula V, for example diethyl 4-(4-substitutedbutoxy)-3,5-dihalobenzenephosphate, can be reacted with 2,2-dialkyl-2,3-dihydrobenzo[b]furan-7-ol (B) in the presence of a base, a solvent and a catalyst at elevated temperature to form a dialkyl 4-[4-(2,2-dialkyl(2,3-dihydrobenzo[2,3-b]furan-7-yloxy))alkoxy]-3,5-dihalobenzenephosphate, a compound of formula IV, for example diethyl 4-[4-(2,2-dialkyl(2,3-dihydrobenzo[2,3-b]furan-7-yloxy))butoxy]-3,5-dihalobenzenephosphate.

In step (c) of Example 2, a dialkyl 4-[4-(2,2-dialkyl(2,3-dihydrobenzo[2,3-b]furan-7-yloxy))alkoxy]-3,5-dihalobenzenephosphate, a compound of formula IV, for example diethyl 4-[4-(2,2-dialkyl(2,3-dihydrobenzo[2,3-b]furan-7-yloxy))butoxy]-3,5-dihalobenzenephosphate, can be reacted as in Example 1 to produce the expected product (I).

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly this invention includes all modifications encompassed within the spirit and scope as defined by the following claims.

What is claimed is:

1. A process for preparing a compound of formula I: 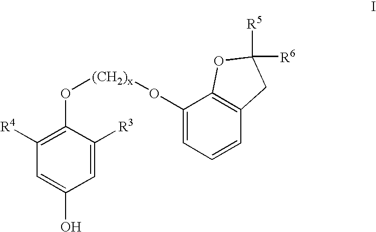
wherein
R³ and R⁴ are selected from halogen;
R⁵ and R⁶ are independently selected from halogen or alkyl; and
x is 2, 3, 4, 5 or 6;
said process comprising:
a) reacting a compound of formula (A): 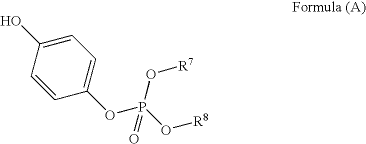
wherein R⁷ and R⁸ are independently selected from hydrogen, alkyl, aryl or R⁷ and R⁸ taken together with an alkyl or aryl, forming a cyclic ester;
with a halogenating agent in the presence of a base to form a compound of formula II: 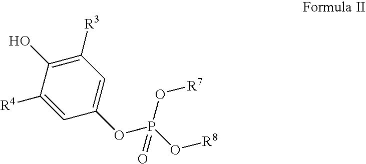
wherein
R³ and R⁴ are as defined above; and
R⁷ and R⁸ are as defined above;
b) reacting a compound of formula (B): 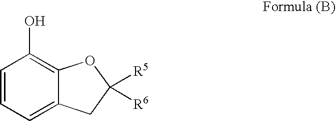
wherein R⁵ and R⁶ are as defined above;
with a compound of formula (C): 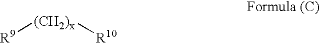
wherein
R⁹ and R¹⁰ are independently selected from halogen, hydroxyl or —OSO₂R¹¹
wherein R¹¹ is alkyl or aryl; and
x is 2, 3, 4, 5 or 6;
in the presence of a base to form a compound of formula III: 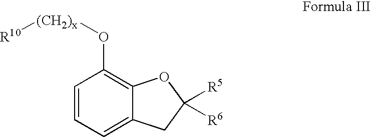
wherein
R⁵, R⁶ R¹⁰ and x are as defined above;
c) reacting a compound of formula II with a compound of formula III in the presence of a base to form a compound of formula IV: 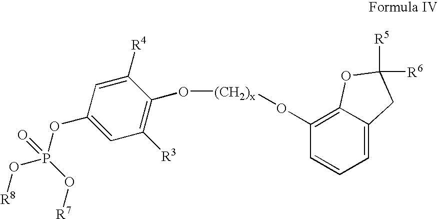
wherein
R³, R⁴, R⁵, R⁶, R⁷, R⁸ and x are as defined above; and
d) reacting a compound of formula IV to form a compound of formula I.

2. The process of claim 1 wherein the reacting of step b) is conducted in the presence of a catalyst.

3. The process of claim 1 wherein the reacting of step b) is conducted at elevated temperature.

4. The process of claim 1 wherein the reacting of step c) is conducted in the presence of a solvent.

5. The process of claim 1 wherein the reacting of step c) is conducted in the presence of a catalyst.

6. The process of claim 1 wherein the reacting of step c) is conducted at elevated temperature.

7. The process of claim 1 wherein the reacting of step d) is with a base.

8. The process of claim 1 wherein the reacting of step d) is a hydrolysis in the presence of an acid.

9. The process of claim 1 wherein the reacting of step d) is conducted in the presence of a solvent.

10. The process of claim 1 wherein the reacting of step d) is conducted in the presence of a catalyst.

11. A process for preparing a compound of formula I: 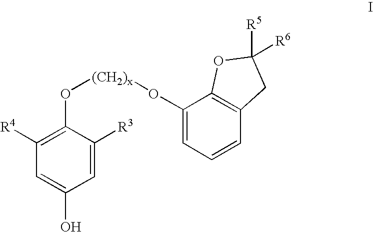
wherein
R³ and R⁴ are selected from halogen;
R⁵ and R⁶ are independently selected from halogen or alkyl; and
x is 2, 3, 4, 5 or 6;
said process comprising:
a) reacting a compound of formula II: 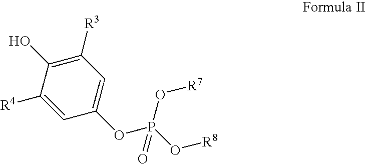
wherein
R³ and R⁴ are as defined above; and
R⁷ and R⁸ are independently selected from hydrogen, alkyl, aryl or R⁷ and R⁸ taken together with an alkyl or aryl, forming a cyclic ester;
with a compound of formula (C): 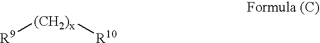
wherein
R⁹ and R¹⁰ are independently selected from halogen, hydroxyl or —OSO₂R¹¹
wherein R¹¹ is alkyl or aryl; and
x is 2, 3, 4, 5 or 6;
in the presence of a base to form a compound of formula V: 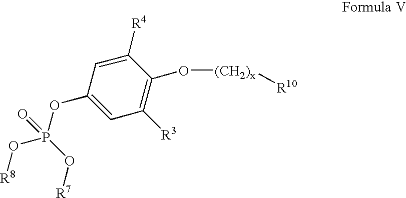
wherein
R³, R⁷, R⁸, R¹⁰ and x are as defined above;
b) reacting a compound of formula V with a compound of formula (B): 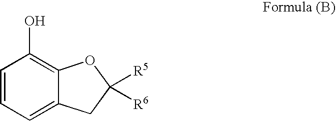
wherein R⁵ and R⁶ are as defined above;
in the presence of a base to form a compound of formula IV: 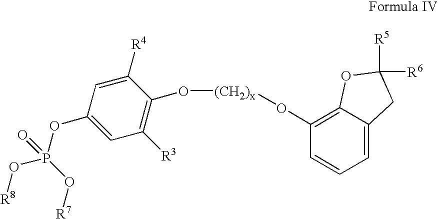
wherein
R³, R⁴, R⁵, R⁶, R⁷, R⁸ and x are as defined above; and
c) reacting a compound of formula IV to form a compound of formula I.

12. The process of claim 11 wherein the reacting of step a) is conducted in the presence of a catalyst.

13. The process of claim 11 wherein the reacting of step a) is conducted at elevated temperature.

14. The process of claim 11 wherein the reacting of step b) is conducted in the presence of a solvent.

15. The process of claim 11 wherein the reacting of step b) is conducted in the presence of a catalyst.

16. The process of claim 11 wherein the reacting of step b) is conducted at elevated temperature.

17. The process of claim 11 wherein the reacting of step c) is with a base.

18. The process of claim 11 wherein the reacting of step c) is a hydrolysis in the presence of an acid.

19. The process of claim 11 wherein the reacting of step c) is conducted in the presence of a solvent.

20. The process of claim 11 wherein the reacting of step c) is conducted in the presence of a catalyst.

21. A compound of formula II: 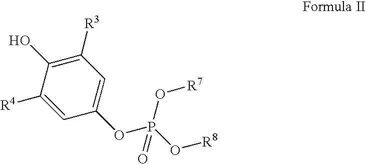
wherein
R³ and R⁴ are selected from halogen; and
R⁷ and R⁸ are independently selected from hydrogen, alkyl, aryl or R⁷ and R8 taken together with an alkyl or aryl, forming a cyclic ester.

22. A compound of formula III: 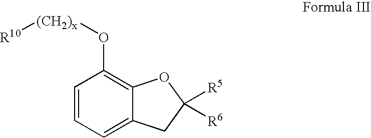
wherein
R⁵ and R⁶ are independently selected from halogen or alkyl;
R¹⁰ is selected from halogen, hydroxyl or —OSO₂R¹¹
wherein R¹¹ is alkyl or aryl; and
x is 2, 3, 4, 5 or 6.

23. A compound of formula IV: 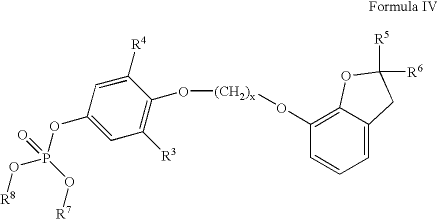
wherein
R³ and R⁴ are selected from halogen;
R⁵ and R⁶ are independently selected from halogen or alkyl;
R⁷ and R⁸ are independently selected from hydrogen, alkyl, aryl or R⁷ and R⁸ taken together with an alkyl or aryl, forming a cyclic ester; and
x is 2, 3, 4, 5 or 6.

24. A compound of formula V: 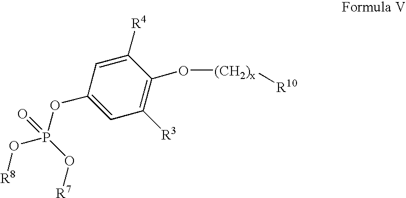
wherein
R³ and R⁴ are selected from halogen;
R⁷ and R⁸ are independently selected from hydrogen, alkyl, aryl or R⁷ and R⁸ taken together with an alkyl or aryl, forming a cyclic ester;
R¹⁰ is selected from halogen, hydroxyl or —OSO₂R¹¹
wherein R¹¹ is alkyl or aryl; and
x is 2, 3, 4, 5 or 6.
